(19) United States
(12) Patent Application Publication
(10) Pub. No.: US 2020/0240980 A1
(43) Pub. Date: Jul. 30, 2020

(54) METHOD FOR DELAYED RECTIFIER CURRENT ENHANCEMENT, CHARACTERIZATION, AND ANALYSIS IN HUMAN INDUCED PLURIPOTENT STEM CELLS

(71) Applicant: The Research Foundation for The State University of New York, Amherst, NY (US)

(72) Inventors: Randall Lee RASMUSSON, Pendleton, NY (US); Glenna C.L. BETT, Albion, NY (US)

(21) Appl. No.: 16/755,588

(22) PCT Filed: Oct. 12, 2018
(86) PCT No.: PCT/US18/55649

§ 371 Date: Apr. 12, 2020

Related U.S. Application Data
(60) Provisional application No. 62/571,483, filed on Oct. 12, 2017.

(51) Int. Cl. G01N 33/50; C12N 5/074; C12N 5/077

(52) U.S. Cl. CPC G01N 33/5061; C12N 5/0661; C12N 5/0696

(57) ABSTRACT

A method for analyzing a test agent to determine whether or not the test agent affects electrical activity of a cardiomyocyte is disclosed. The method includes: i) providing an induced pluripotent stem cell (iPSC) derived cardiomyocyte (iPSC-DM) that has been modified to contain an oligonucleotide that increases iPSC-DM I_{Kr}; ii) measuring I_{Kr} and action potential of the iPSC-DM of i); subsequently iii) contacting the iPSC-DM of i) with the test agent; and iv) measuring I_{Kr} and action potential of the iPSC-DM that has been contacted with the test agent; and determining a difference between the I_{Kr} and action potential of ii) and the I_{Kr} and the action potential of iv) to indicate the test agent affects the electrical activity of the cardiomyocyte, or determining the same I_{Kr} and action potential of ii) and iv) to indicate the test agent does not adversely affect the electrical activity of the cardiomyocyte.

### CROSS REFERENCE TO RELATED APPLICATIONS

This application claims priority to U.S. Provisional Application No. 62/571,483, filed on Oct. 12, 2017, the disclosure of which is hereby incorporated by reference.

### STATEMENT REGARDING FEDERALLY SPONSORED RESEARCH

This invention was made with government support under contract no. NIH R41HL-127901 awarded by the National Institutes of Health. The government has certain rights in the invention.

### FIELD OF THE DISCLOSURE

The present disclosure relates to drug screening, specifically screening for drugs producing proarrhythmic side effects.

### BACKGROUND OF THE DISCLOSURE

The delayed rectifier current, I_{Kr}, plays a fundamental role in cardiac electrical function, and is a major determinant of cardiac health. Abnormalities in I_{Kr} can result in prolongation of the cardiac action potential (AP) or shortening of the AP, both of which are proarrhythmic. In addition to the profound clinical significance of the intrinsic properties of I_{Kr}, mutations in I_{Kr} underlie the gene LQT2, and modification of I_{Kr} is also the major mediator of drug-induced arrhythmias. Pharmacological interaction of an incredibly wide range of pharmacological agents with I_{Kr} can lead to torsade de pointes, and subsequently potentially fatal arrhythmias.

Given the enormous significance of I_{Kr}, it is surprising that so little is known about human cardiac I_{Kr}. The majority of knowledge about human I_{Kr} has been inferred from voltage clamp experiments on heterologous expressions of HERG1a, which is only one putative isoform of the channel complex underlying I_{Kr}. HERG1a does not completely match the few kinetic datasets on the behavior of human I_{Kr} recorded from healthy freshly isolated human ventricular myocytes, and the behavior of HERG1a is sensitive to the system in which it is expressed. Recent evidence points to a role for HERG1as well as HERG1a in human cardiac myocytes.

I_{Kr} has unusual gating characteristics which require complex and lengthy voltage clamp protocols which are incompatible with the fragility and limited availability of freshly isolated healthy human ventricular myocytes. Other options, such as animal models and heterologously-expressed systems, are also insufficient. Accordingly, there is a long-felt need for a method to reliably measure, analyze, and characterize I_{Kr} in human cells.

### BRIEF SUMMARY OF THE DISCLOSURE

Compositions, methods and systems for assessing properties of test agents are provided. The test agents can be any type of agent, and the safety of the agents can be assessed for, among other parameters described herein, whether or not the test agent alters an action potential or other electrical property of cardiac myocytes, and/or causes or promotes development of pro-arrhythmic properties.

In an embodiment, the disclosure comprises a method for analyzing a test agent to determine whether or not the test agent affects electrical activity of a cardiomyocyte. The method comprises:

i) providing an induced pluripotent stem cell (iPSC) derived cardiomyocyte (iPSC-DM) that has been modified to contain an oligonucleotide that increases iPSC-DM I_{Kr};

ii) measuring I_{Kr} and action potential of the iPSC-DM of i); subsequently

iii) contacting the iPSC-DM of i) with the test agent; and

iv) measuring I_{Kr} and action potential of the iPSC-DM that has been contacted with the test agent; and determining a difference between the I_{Kr} and action potential of ii) and the I_{Kr} and the action potential of iv) to indicate the test agent affects the electrical activity of the cardiomyocyte, or determining the same I_{Kr} and action potential of ii) and iv) to indicate the test agent does not adversely affect the electrical activity of the cardiomyocyte.

In embodiments, the method includes determining a value of an inward rectifying current (I_{K1}) for the iPSC-DM of ii) and I_{K1} of the iPSC-DM of iv).

In embodiments, the method further comprises applying the determined I_{K1} to the iPSC-DM, and determining the presence or absence of pro-arrhythmic properties in the iPSC-DM. The presence of pro-arrhythmic properties in the iPSC-DM after treatment with the test agent, and by comparison to the properties before the treatment, indicates the test agent affects electrical activity of cardiomyocytes, and vice versa. In certain implementations the pro-arrhythmic properties that are determined comprise any of: early after depolarizations, action potential prolongation, delayed after depolarizations, steepness of restitution, and action potential triangulations or other in-vitro indices of development of a pro-arrhythmic substrate that will be known to those skilled in the art.

In embodiments, the method includes determining I_{Kr} and action potential for a plurality of iPSC-DMs in a series of distinct samples of the iPSC-DMs. Thus, individual whole cell analysis can be performed in series or concurrently, using distinct whole cells in distinct samples. The method further includes determining I_{Kr} for the plurality of the iPSC-DMs and applying a calculated I_{K1} to the plurality of iPSC-DMs in the series of distinct samples, and determining the presence or absence of pro-arrhythmic properties therein.

In an embodiment, the disclosure comprises measuring the I_{Kr} and action potential of the iPSC-DM of ii) to characterize I_{Kr} and the action potential as either slowly deactivating-type (HERG1a-like) or rapidly deactivating-type (HERG1b-like).

### DESCRIPTION OF THE DRAWINGS

For a fuller understanding of the nature and objects of the disclosure, reference should be made to the following detailed description taken in conjunction with the accompanying drawings.

FIG. 1 is a series of three plots of tail currents for three I_{Kr} sub-types: I_{KrB} (top), I_{KrA} (middle), and I_{KrA,B} (bottom). This plot shows each sub-type's kinetically distinct rate of deactivation. The white line through I_{KrB} and I_{KrA} is the single exponential least squares fit for the tail current plots. The white line through I_{KrA,B} is the double exponential least squares fit for the tail current plot.

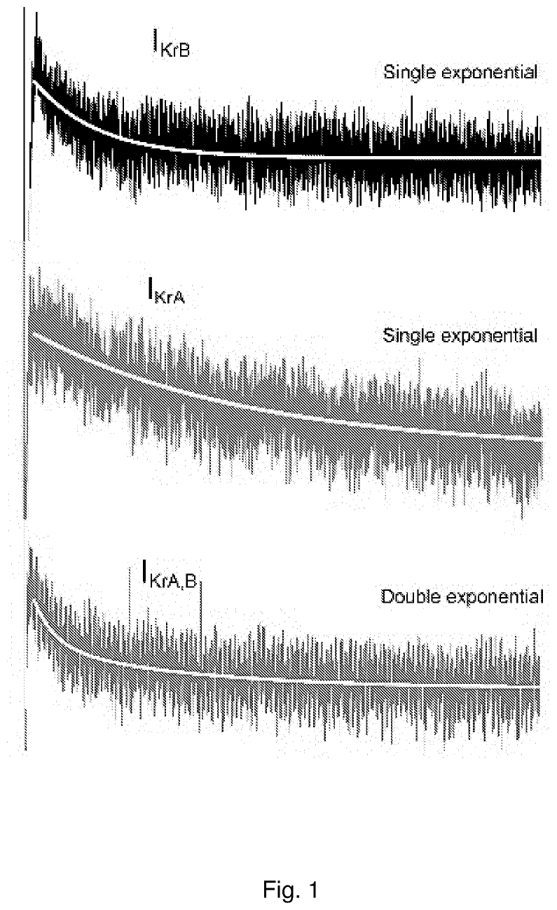

FIG. 2 is a series of three action potential plots showing how the injection of I_{K1} into h-iPSCD cardiocytes enables cells to be identified as atrial or ventricular. The top pair of plots shows how the injected I_{K1} restores resting potential, and spike and dome behavior is observed in ventricular cells. The middle pair of plots shows the action potential of atrial cells stabilized with I_{K1} injection. The bottom pair of plots shows the action potential of atrial and ventricular cells on an expanded timescale.

FIG. 3 shows an expression of HERG protein which is abundant on the epicardium, but largely absent from mid and endocardium. The HERG protein is also more abundant at the apex (EE), but becomes sparser towards the base (GG).

FIG. 4 is a graph showing the distribution of I_{Kr} current densities ≤0.2 pA/pF or >0.2 pA/pF for hiPSCDCMs treated with morpholino AS or naïve/vehicle cells. The graph shows a significant shift (p<0.05) in I_{Kr} current densities >0.2 pA/pF for morpholino AS-treated cells.

FIG. 5 contains current plots (A and B) showing I_{Kr} in hiPSCDCMs, more specifically I_{Kr} activation current in vehicle-treated hiPSCDCMs in the absence (A) and presence (B) of 1 μM dofetilide. FIG. 5 also contains a current-voltage curve plot (C) for P2 peak tail current for naïve/vehicle-cells and dofetilide (0.3 to 3 μM)-treated hiPSCDCMs, where P2 is a single voltage step of −50 mV. In A and B, the red trace is a +30 mV P1 pulse, where P1 consists of a 10 mV voltage step every 4 s between −80 and +50 mV. Note the native currents flowing during the P1 pulse and the well isolated, dofetilide sensitive, I_{Kr} tail current during P2.

FIG. 6 shows two current plots of iPSCDCM I_{Kr} having a HERG1a-like phenotype and a

HERG1b-like phenotype during deactivation. The black lines represent I_{Kr}, while the red lines represent heterologously expressed HERG1a or 1b currents.

FIG. 7 is a histogram showing two distinct populations of fast and slow deactivation in stem cell derived cardiac myocytes.

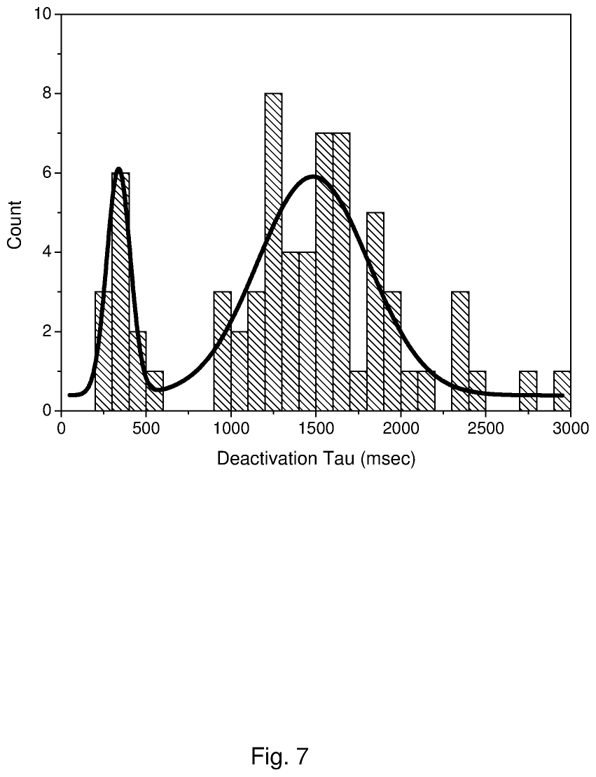

FIGS. 8A and 8B show restitution curves (APD₉₀) for two types of ventricular I_{Kr}. FIG. 8A shows restitution with a fast deactivating I_{Kr}, and FIG. 8B shows restitution with a slow deactivating I_{Kr}.

FIG. 9 is a flowchart depicting a method according to a non-limiting embodiment of the present disclosure. In FIG. 9, the control IPSC-derived cardiac myocyte that has been contacted with morpholino anti-sense oligonucleotides and has been characterized as the same I_{Kr}-type as the test cell can be the same test cell, where the I_{Kr} was determined prior to being contacted with the test agent.

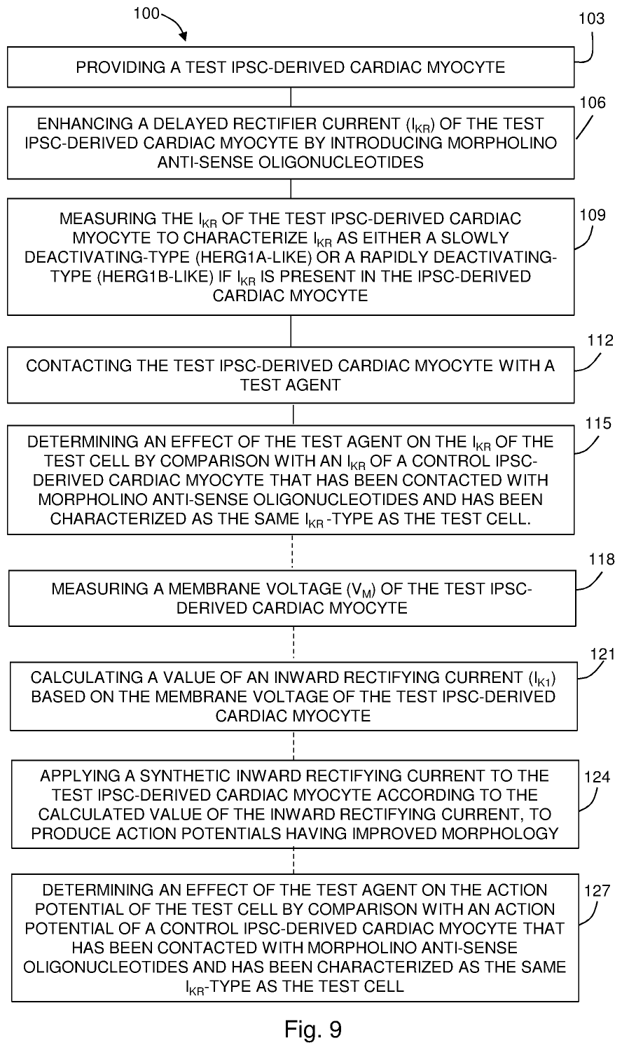

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various changes may be made without departing from the scope of the disclosure.

Unless defined otherwise herein, all technical and scientific terms used in this disclosure have the same meaning as commonly understood by one of ordinary skill in the art to which this disclosure pertains.

Ranges of values are disclosed herein. Unless otherwise stated, the ranges include all values to the magnitude of the smallest value (either lower limit value or upper limit value) and ranges between the values of the stated range. The disclosure includes practicing embodiments of the disclosure with any of devices and systems described herein, all time periods, temperatures, media, buffers, and other compositions of matter that are described herein. The disclosure includes all steps and all combinations of steps of the methods described herein, some of which may be performed concurrently, or sequentially. Certain steps as will be apparent from the description may be omitted.

All modified cells as described herein, including, but not necessarily limited to such cells being in contact with one or more test agents, are included in this disclosure.

The disclosure includes all polynucleotide sequences described herein, their complementary sequences, and reverse complementary sequences. The disclosure includes sequences that share sequence identity with the described sequences, provided the intended function of the molecule comprising or consisting of such sequences is maintained. Unless otherwise indicated, nucleic acids are written left to right in 5′ to 3′ orientation.

In an aspect of the present disclosure, a method of identifying a test agent for drug development is presented. The test agent is not particularly limited, and can be, for example, any drug or other small molecule, a protein, a peptide, a polynucleotide, or an antibody. The test does not require prior knowledge of any function of the test agent. In embodiments, a combination of test agents can be analyzed. In embodiments, a method of identifying a test agent comprises determining a safety profile for the test agent. In embodiments, the safety profile comprises determining whether or not the test agent alters an action potential or other electrical property of cardiac myocytes, and/or causes or promotes development of pro-arrhythmic properties, as further described below. In embodiments, the disclosure comprises analysis of a single cell. In embodiments, a series of single cell measurements is made. In embodiments, an average value or other indicator from a series of measurements is produced. In embodiments, single cell measurements are made in a series of distinct samples. The samples may comprise cells and cell cultures that will be readily apparent to those skilled in the art, given the benefit of this disclosure, and include samples of pluralities of cells in any suitable culture plate, dish, cover slip, etc.

Cells that are used in embodiments of this disclosure comprise induced pluripotent stem cell (iPSC) derived cardiomyocytes, also referred to herein as iPSC-DMs, cardiac myocytes, and iPSCDCMs. iPSC-DMs, and methods of making them, are known in the art, and suitable iPSC-DMs are commercially available.

The test agent is identified for drug development by analyzing an enhanced I_{Kr} of a test iPSC-derived cardiac myocytes, wherein the enhanced property of the iPSC-derived cardiac myocyte or a population thereof represents the I_{Kr} of healthy cardiac myocytes. The method 100 includes providing 103 a test iPSC-derived cardiac myocyte. The method uses iPSC-derived cardiac myocytes because native human myocytes are not available in quantities suitable for I_{Kr} or action potential analysis.

As described above, the disclosure relates in certain embodiments to determining a safety profile for the test agent, and can include determining whether or not the test agent alters an action potential or other electrical property of cardiac myocytes, and/or causes or promotes development of pro-arrhythmic properties, as further described below. Accordingly, aspects of the disclosure relate to using cells having enhanced I_{Kr} for assessing safety profiles of drugs that may affect the heart. Thus, the presently provided assay, based on the kinetics and behavior of human I_{Kr}, is expected to improve drug development and increase the ability to identify drugs with the potential to have significant adverse events. In this regard, all drugs for which U.S. Food and Drug Administration (FDA) approval is sought are required to demonstrate comprehensive pre-clinical safety testing. This testing is required regardless of the target of the candidate drug, the mode of action, or the disease being treated. Screening is required to ensure the drug does not have adverse effects on the electrical activity of the heart. For example, the FDA requires demonstration that every new investigational compound does not induce arrhythmogenic, and potentially fatal, prolongation of the QT interval in humans QT prolongation increases the likelihood of the potentially fatal Torsade de Pointes arrhythmia, and is the most common reason for an already-marketed drug being withdrawn or relabeled. FDA applications must demonstrate the drug neither prolongs QT nor blocks I_{Kr}, such as by inhibiting current flow through a channel by direct interaction with the channel, including but not limited to physically obstructing ions from flowing through the channel

Most previously available methods of drug safety screening are inaccurate, expensive, and result in significant false positive and false negative errors. Fresh human cardiac cells are not available in quantities suitable for preclinical testing, so a variety of animal models and heterologously-expressed systems are used. Heterologous expression systems are known to be insufficient, perhaps due to a lack of intracellular components (ancillary subunits, etc.,) that can profoundly alter drug binding properties. The properties of I_{Kr} in animal models are highly variable between species, in part due to variations in the molecular basis of I_{Kr}. This assay uses an important and previously unsuspected, property of I_{Kr} in human cells. It is believed to be impossible to carry out this assay in native human myocytes, and so iPSCD cardiac cells offer a unique human assay for voltage clamp analysis.

The FDA is actively seeking improved drug safety testing paradigms, and has specifically requested development of new approaches which combine computer models and human induced pluripotent stem cell derived cardiac myocytes. The FDA recognizes the importance of improving the process of drug development and safety testing, and they issued a recent statement concerning a new initiative, emphasizing the need to make the process more efficient, and to move the major clinical/regulatory analysis concerning arrhythmogenic potential earlier in the drug discovery and development continuum, enhance the accuracy with which existing and/or new drugs are labelled relative to actual proarrhythmic risks, and increase the output of new chemical entities that benefit patients. In addition, the FDA recommends cellular systems, combined with integrated computer models, and “confirmation of the electrophysiological effects in a myocyte assay such as human induced pluripotent stem cell-derived cardiomyocytes.” The presently provided enhanced I_{Kr} assay enables human iPSCDCMs to be used as a precise and accurate investigational tool for preclinical drug testing.

The majority of drug development assays and FDA mandated drug safety screening is undertaken on the interaction of the drug with heterologously expressed HERG1a. There are significant differences between the pharmacological and pathophysiological profile of HERG1a and I_{KrA}, I_{KrB}, and I_{KrA/B}. The new approach of this disclosure transforms the current approach to drug development and safety screening, and will reduce the number of drugs with serious adverse events. While this new approach focuses on voltage clamp methods for testing, other systems such as optical methods or micro-electrode arrays also suffer from high variability due to inconsistent HERG expression. Consistent and robust native I_{Kr} expression will also significantly improve the homogeneity, responsiveness, and reliability of multicellular systems.

Thus, in embodiments, the method includes enhancing 106 I_{Kr} and/or action potential of the test iPSC-derived cardiac myocyte by introducing (e.g., by transfection or any other suitable approach) an oligonucleotide that functions in an anti-sense and/or RNAi-mediated manner This enhancement is desirable as I_{Kr} has a very small magnitude in a large number of cells that are used in previously available methods.

In more detail, in certain embodiments, the disclosure relates to use of an oligonucleotide to affect production of isoforms of RNA produced by the hERG1 gene in iPSC-DMs. This gene is also known as KCNH2. hERG1 encodes the Kv11.1 channel, which is known to conduct the rapidly activating delayed rectifier K+ current (I_{Kr}) in certain heart cells. Isoforms of the HERG1 protein are known in the art and are referred to as KV11.1a, which is functional, and KV11.1a-USO, which is non-functional. Expression of the non-functional KV11.1a-USO is due to the presence of a specific poly(A) signal in Intron 9. In embodiments, the disclosure thus uses an oligonucleotide that targets a sequence in an RNA that is required for polyadenylation of intron 9 of the RNA transcribed from hERG1 gene, such polyadenylation resulting in production of non-functional HERG1. Hybridization of the oligonucleotide may therefore reduce polyadenylation, and/or influence splicing of the RNA, and/or inhibit translation of the RNA, and/or promote degradation of the RNA. In embodiments, an oligonucleotide anneals to and/or eliminates the poly(A) signal in Intron 9, preventing or inhibiting expression of the non-functional KV11.1a-USO and allowing for the preferential expression of the functional KV11.1a potassium channel variant. The net result is an increase in current levels for the KV11.1 potassium channel

In embodiments, the oligonucleotide used in methods of this disclosure comprises a modified oligonucleotide. In embodiments, the oligonucleotide may be a DNA analog. The DNA analog may include modified nucleotides and/or modified nucleotide linkages. Suitable modifications and methods for making DNA analogs are known in the art. Some examples include but are not limited to polynucleotides which comprise modified ribonucleotides or deoxyribonucleotides. For example, modified ribonucleotides may comprise methylations and/or substitutions of the 2′ position of the ribose moiety with an —O— lower alkyl group containing 1-6 saturated or unsaturated carbon atoms, or with an —O-aryl group having 2-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In embodiments modified nucleotides comprise methyl-cytidine and/or pseudo-uridine. The nucleotides may be linked by phosphodiester linkages or by a synthetic linkage, i.e., a linkage other than a phosphodiester linkage. Examples of inter-nucleoside linkages in the polynucleotide agents that can be used in the disclosure include, but are not limited to, phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof. In embodiments, the DNA analog may be a peptide nucleic acid (PNA). In embodiments, the oligonucleotide is a morpholino oligonucleotide, an example of which comprises morpholino anti-sense (AS) oligonucleotides. In embodiments, the oligonucleotide is conjugated to a moiety, protein, or peptide, which facilitates cell and/or nuclear penetration. In an embodiment, the oligonucleotide is conjugated to a weak-base amphiphilic peptide known in the art as Endo-Porter (see, for example, Ann N Y Acad Sci. 2005 November; 1058:62-75). Non-limiting examples of the disclosure are illustrated using morpholino anti-sense (AS) oligonucleotides. Where reference is made to a morpholino it refers to a morpholino AS oligonucleotide.

In embodiments, the morpholino AS oligonucleotide is specific for a polyadenylation signal in Intron 9 of the KCNH2 gene, which codes for the potassium channel Kv11.1 (also known as HERG). In embodiments, the length of the morpholino AS or other modified oligonucleotides may range from 20 to 30 bases. In one example, the size of the morpholino AS oligonucleotides may be 25 bases. In embodiments, the morpholino or other modified oligonucleotide comprises or consists of the sequence 5′-CAGAACACAGTAGTGAATCAAAACC-3′ (SEQ ID NO:1), referred to herein as alpha2NS 05Z. In embodiments, the oligonucleotide comprises a contiguous segment of SEQ ID NO:1 that is at least 20 nucleotides in length. In embodiments, the contiguous segment of SEQ ID NO:1 contains in a 5′−3′ direction the sequences ACACA and CAAAACC.

In non-limiting examples, contacting the cells described herein with the morpholino anti-sense oligonucleotides increases the current density of I_{Kr}. In an embodiment, the iPSC-derived cardiac myocyte may be in contact with morpholino anti-sense oligonucleotides for approximately 48 hours, and changes remain viable for up to ten days, but other time periods can be used. In embodiments, the cardiac myocytes are in contact with the morpholino for a period of 12-48 hours, inclusive, and including all intervals of time between 12-48 hours.

Embodiments of the disclosure further include measuring 109 the I_{Kr} of the test iPSC-derived cardiac myocyte to characterize I_{Kr} as either a slowly deactivating-type (HERG1a-like) or a rapidly deactivating-type (HERG1b-like) if I_{Kr} is present in the iPSC-derived cardiac myocyte. Since I_{Kr} is not present in a large number of cells, the cells with I_{Kr} insufficient for characterization are removed from the analysis process. Characterizing the I_{Kr} as slowly deactivating or rapidly deactivating allows accurate selection of a corresponding type of control cell for comparison in subsequent steps. In an example of this aspect of the present disclosure, I_{Kr} may be measured by a patch clamp. In a further example, the test iPSC-derived cardiac myocyte is clamped at −80 mV with currents recorded over a 4 s voltage step from the holding potential to potentials between −80 and +50 mV in increments of 10 mV.

The method further includes contacting 112 the test iPSC-derived cardiac myocyte with a test agent. The method further includes determining 115 an effect of the test agent on the I_{Kr} and/or the action potential of the test cell by comparison with an I_{Kr} and/or action potential of a control iPSC-derived cardiac myocyte that has been contacted with morpholino anti-sense oligonucleotides and has been characterized as the same I_{Kr}-type and/or action potential as the test cell. In this regard, as is known in the art, I_{Kr} current is a critical component of the action potential cells, and any modification of I_{Kr} has a significant effect on the action potential shape and duration. Accordingly, in embodiments, properties of cells as described herein comprise integrated properties of the cell. Thus, in embodiments, the effect of I_{Kr} current is not analyzed directly, rather the effects of I_{Kr} expression on the whole cell system are analyzed.

The determination of the effect of the test agent may involve analysis of any cellular property described herein or otherwise known to those skilled in the art. In embodiments, the disclosure comprises determining one or any combination of pro-arrhythmic properties displayed by the test cell, examples of which include but are not limited to, early after depolarizations, action potential prolongation, delayed after depolarizations, steepness of restitution, and action potential triangulations.

In an example of this aspect of the present disclosure, the method may further include placing the iPSC-derived cardiac myocyte in a solution, such as a physiological solution, to carry out any one or combination of steps described herein. In embodiments, methods described herein preserves the ability of iPSCDCMs to achieve consistent GΩ seals for patch clamp use.

An aspect of the present disclosure includes steps for analyzing the action potential of the test iPSC-derived cardiac myocyte which has been contacted with morpholino anti-sense oligonucleotides. Analyzing the action potential of iPSC-derived cardiac myocytes and other electrical properties of such cells is described in U.S. Patent Publication 20160083715, published on Mar. 24, 2016, the entire disclosure of which is incorporated herein by reference. The steps may include measuring 118 a membrane voltage (Vₘ) of the test iPSC-derived cardiac myocyte. In an example of this aspect of the present disclosure, Vₘ may be measured by a patch clamp. The patch clamp may be used in a whole-cell configuration to measure Vₘ. The disclosure thus provides for use of iPSCDCM action potentials for validation of drug studies, by bypassing the expensive Thorough-QT (TQT) requirements currently in place. However, the depolarized and spontaneous nature of the unmodified iPSCDCM action potential limits the ability to perform standard measures of pro-arrhythmic changes, such as steepness of the restitution curve. By combining the presently provided molecular approach, analysis, and quality control with dynamic clamp mediated “electronic expression” of I_{K1} we can stabilize and normalize the action potential.

Additional steps further include calculating 121 a value of an inward rectifying current (I_{K1}) based on the membrane voltage of the test iPSC-derived cardiac myocyte. In an example of this aspect of the present disclosure, I_{K1} may be calculated from the membrane voltage according to the equation:

$${I}_{K 1} = 0.5 ( \frac{{V}_{m} + 85}{1 + {e}^{0.0896 ( {V}_{m} + 85 )}} ) + 0.01 ( {V}_{m} + 85 ) . \qquad ( 1 )$$

Additional steps further include applying 124 a synthetic inward rectifying current to the test iPSC-derived cardiac myocyte according to the calculated value of the inward rectifying current, to produce action potentials having improved morphology, such as stable resting potentials, “spike and dome” response characteristics, and rapid sodium mediated upstrokes. The synthetic inward rectifying current corresponds to the calculated 121 value. In an example of this aspect of the present disclosure, the synthetic inward rectifying current may be applied to the iPSC-derived cardiac myocyte by a patch clamp. In a further example of this aspect of the present disclosure, the applied 124 synthetic current is less than the calculated 121 value, for example, where a potentiometer is used to attenuate the current.

The additional steps can further include determining 127 an effect of the test agent on the action potential of the test cell by comparison with an action potential of a control IPSC-derived cardiac myocyte that has been contacted with morpholino anti-sense oligonucleotides and has been characterized as the same I_{Kr}-type as the test cell, which may in fact be the same cell. Thus, the disclosure comprises testing the same cell twice, once before it is contacted with the test agent, and then after it has been contacted with the test agent, to determine any parameter described herein. Likewise, a plurality of cells can be tested, wherein distinct cells from distinct samples are tested.

It is in view of the foregoing that the following certain aspects of this disclosure are characterized, as follows.

Similar to freshly isolated human cardiac tissue, both HERG1a and HERG1b are expressed in iPSCDCM I_{Kr}. iPSCDCMs therefore offer a unique I_{Kr} assay in a native human cardiac environment.

Three kinetically-distinct dofetilide-sensitive types of I_{Kr} in human ventricular myocytes have been identified. These currents have kinetics which are consistent with a solely HERG1a-based (i.e., slowly deactivating current, I_{KrA}) current, a solely HERG1b-based (i.e., rapidly deactivating current, I_{KrB}) current, and a current with both fast and slow deactivation components which are consistent with non-interacting HERG 1 a and 1b contributions (I_{KrA/B}). The plentiful availability of stable iPSCDCMs offers a unique way to form a human ventricular I_{Kr} assay.

Human I_{Kr} has unique electrophysiological properties, and unique roles in rate and drug-dependent dispersion of repolarization. The molecular and kinetic heterogeneity of I_{KrA}, I_{KrB}, and I_{KrA/B} has profound implications for I_{Kr} as a determinant of physiological and pathophysiological repolarization and restitution as well as for pharmacology, drug screening, and safety testing.

Importance of I_{Kr} in Cardiac Physiology and Pathophysiology

I_{Kr} plays an important role in the timing of repolarization of the cardiac AP. Consequently, the restitution portrait of cardiac repolarization is strongly dependent on I_{Kr}, and changes in I_{Kr} can be strongly proarrhythmic. AP prolongation is proarrhythmic, but AP duration is not the only important quantity; the shape as well as duration can be critical in determining arrhythmogenic potential. Both the duration and shape of repolarization is critical for determining the propensity for setting up reentrant arrhythmias. Any abnormality that increases, either temporally or spatially, the duration or physical size of the “vulnerable window” tends to make induction of tachycardia by an ill-timed impulse more likely. The importance of AP shape on the arrhythmogenic potential of cardiac muscle is clearly evident in the vulnerable window mechanism. However, the full importance of AP shape may not yet be fully realized. In addition to the N-terminal defects which alter the kinetics of HERG, other mutations in HERG have been found which lead to congenital short QT syndrome. Surprisingly, the short QT defect is also associated with arrhythmias and has a poor patient prognosis. Overall, little correlation has been found between patient longevity, morbidity, and the severity of QT prolongation, but the available data suggest that arrhythmogenesis is strongly dependent on I_{Kr} kinetics. The differing kinetics of I_{KrA}, I_{KrB} and I_{KrA,B} are therefore likely to have a strong influence on arrhythmogenesis.

HERG (without discrimination of isoform) has substantial differential expression both transmurally, and from apex to base. The data demonstrates that human I_{KrA}, I_{KrB}, and I_{KrA,B} have orders of magnitude differences in deactivation kinetics, which are associated with similarly significant changes in the steepness of the fast and slow components of their respective restitution curves. The non-uniform distribution of HERG isoforms, coupled with the non-uniform kinetics of I_{KrA}, I_{KrB}, and I_{KrA,B} will result in substantial heterogeneity of cellular repolarization. I_{KrA}, I_{KrB} and I_{KrA,B} have very different sensitivities to drugs and changes in pH. This uneven sensitivity to frequency and drugs provides an additional mechanism for understanding the arrhythmogenic potential of HERG mutations, as well as pharmacological block of HERG.

Addressing Previous Barriers

Without intending to be constrained by any particular theory, it is considered that the two most prominent reasons for the existing deficit in data from human ventricular I_{Kr} are: (1) the difficulties associated with recording I_{Kr} in human cardiac myocytes; (2) the complex gating of I_{Kr}, which is unusual, and involves both Vₘ-dependent and Vₘ-independent steps. Freshly isolated non-diseased human cardiac tissue suitable for electrophysiology is very difficult to obtain, and there are only very limited reports on I_{Kr}, with even less data on deactivation rates. To resolve this issue, and as described above, an assay on I_{Kr} in iPSCD ventricular myocytes is performed. Assays can be performed on hiPSCDCMs continuously, and I_{Kr} can be recorded and the molecular basis for its kinetic and molecular diversity elucidated. The modified cells described herein offer a unique assay with the various I_{Kr} subtypes (I_{KrA}, I_{KrB}, and I_{KrA/B}) in their native setting of the human ventricular myocyte.

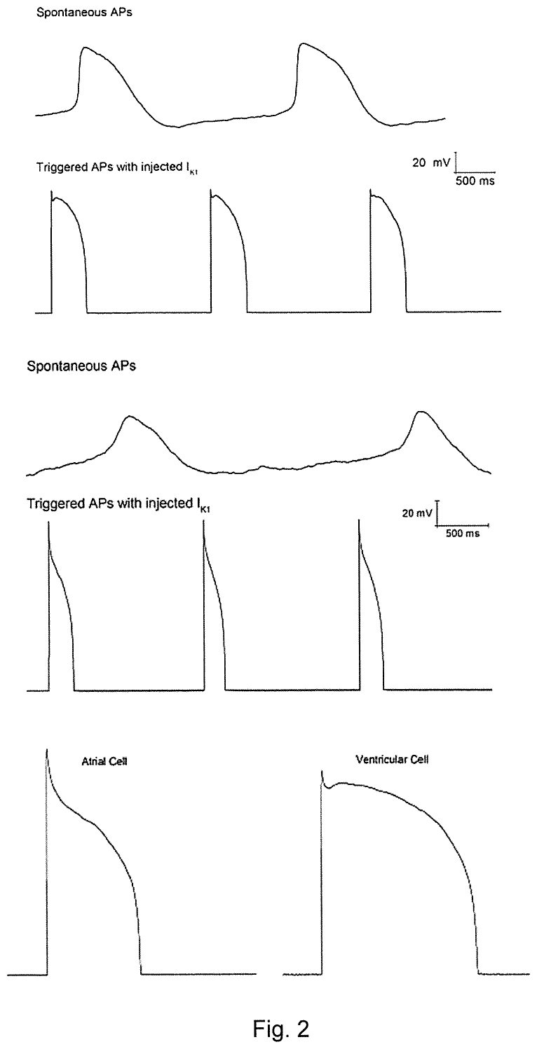

In embodiments, I_{K1} is electronically expressed in iPSCD myocytes via dynamic clamps. This gives these cells a stable physiological resting potential, and cells have readily identifiable APs with a phenotypical spike and dome AP shape as shown in FIG. 2.

New Understanding of Cardiac I_{Kr} Having Kinetically and Pharmacologically Distinct Components: I_{KrA}, I_{KrB}, and I_{KrA,B}.

The inwardly rectifying current, I_{Kr}, was once regarded as a single current. Subsequently, it was determined to have 2 components, I_{Kr}, and I_{Ks}, with distinct kinetics, pharmacology, and molecular basis. There is very little data on human I_{Kr}. The rate of deactivation of human ventricular I_{Kr} deactivation is rarely analyzed, and the few references which report a value for deactivation report data from 2-10 cells. Data from I_{Kr} currents in 88 iPSCD ventricular myocytes revealed that human I_{Kr} has 3 electrophysiologically distinct subtypes. These 3 I_{Kr} components (I_{KrA}, I_{KrB}, and I_{KrA,B}) have clearly distinguishable physiology and pharmacology. Uneven distribution of I_{KrA}, I_{KrB}, and I_{KrA,B} throughout the heart will result in alteration in dispersion of repolarization, and will affect restitution. The various I_{Kr} subtypes have different sensitivity to drugs and to environmental changes such as changes in pH, and so understanding these components is vital to understanding native function of human I_{Kr} in the heart.

Human Ventricular iPSCDCMs

As described above, the presently provided assay is performed on human I_{Kr} in human iPSCD ventricular cells, not an animal model, or an overexpression of a putative part of the channel in a heterologous system. The assay uses high grade commercially-obtained iPSCD cells. These cells are easy to handle, and are consistent and robust cardiac cells. The vials supplied by a commercial supplier are a mixture of ventricular and atrial myocytes. We use the disclosed apparatus, which electronically expresses I_{K1} in iPSCDCMs, and returns the resting membrane potential to physiological levels. This makes the cells quiescent, with physiological resting potentials, and enables atrial and ventricular myocytes to be clearly distinguished as shown in FIG. 2. In embodiments, only ventricular myocytes are used in this assay. For assays with stimulated APs, I_{K1} is electronically expressed, scaled by cellular capacitance, so that ventricular APs can be stimulated from a physiological resting potential.

How I_{Kr} is Measured Using the Disclosed Apparatus

The following description provides a non-limiting approach to implementing methods described herein.

Initially, I_{Kr} is measured with the disclosed apparatus off. Once the magnitude of I_{Kr} is known, the disclosed apparatus is turned on. With the disclosed apparatus on, factors which are greatly affected by the presence of I_{Kr} are then measured and interpreted, including action potential shape and duration, as well as restitution, prolongation, and triangulation.

Representative parameters regarding the measurement are as follows.

First, I_{Kr} is not present, or is only a very small current, in a large number of cells. In the absence of the present disclosure, this gives incorrect or highly variable and therefore undependable results in many cell based assays, including patch clamp, optical methods, and microelectrode arrays.

Second, this variability is due to the nature of the heart and the cells representing a naturally uneven distribution in the ventricular muscle.

Third, in order for the assay to be interpretable, the size of the I_{Kr} current in physiological solutions so that action potentials can be recorded has to be determined. In certain approaches this involves altering the cell with various blockers or using dofetilide sensitive difference currents. In embodiments, I_{Kr} size and type can be identified prior to measurement without using drug-sensitive difference currents.

Fourth, the presently provided assay is advantageous because the precise contribution of HERG to the I_{Kr}-mediated current in native myocytes is unknown. What is clear is that HERG expressed in cloned systems has a different electrophysiological profile and drug sensitivity compared to native I_{Kr}.

Fifth, native I_{Kr} has two components, which are consistent with two different forms of HERG (HERG1a and HERG1b). Only HERG1a is used in heterologously expressed drug safety screening systems. HERG1a and HERG1b have different electrophysiological profiles and drug sensitivities to each other.

Sixth, a kinetic method of identifying the components of I_{Kr} and relating them to action potentials using the disclosed apparatus is described. This is notable because the induced pluripotent cardiac myocyte preparation includes use of the electronic expression of I_{K1} in order to show proper state-dependent drug binding.

Seventh, in order to make the cells more consistent as an assay, we have used a unique approach in cloned channels to increase consistency of the expression of I_{Kr} (HERG) as a measureable current. This method does not result in non-physiological over expression. This greatly reduces variability and increases responsiveness to the known pro-arrhythmic specific HERG channel block. Thus, the disclosed approach of targeting an alternative splice site results in a unique “gentle” manipulation of HERG that results in a functionally relevant upregulation of the current.

This disclosure is therefore in certain embodiments a method for reliably increasing I_{Kr} expression in human iPSCDCMs, and for using this enhanced current for a better prediction of the arrhythmogenicity of a candidate drug. This approach combines molecular manipulation, electrophysiology, and dynamic clamp using the disclosed apparatus dynamic clamp system.

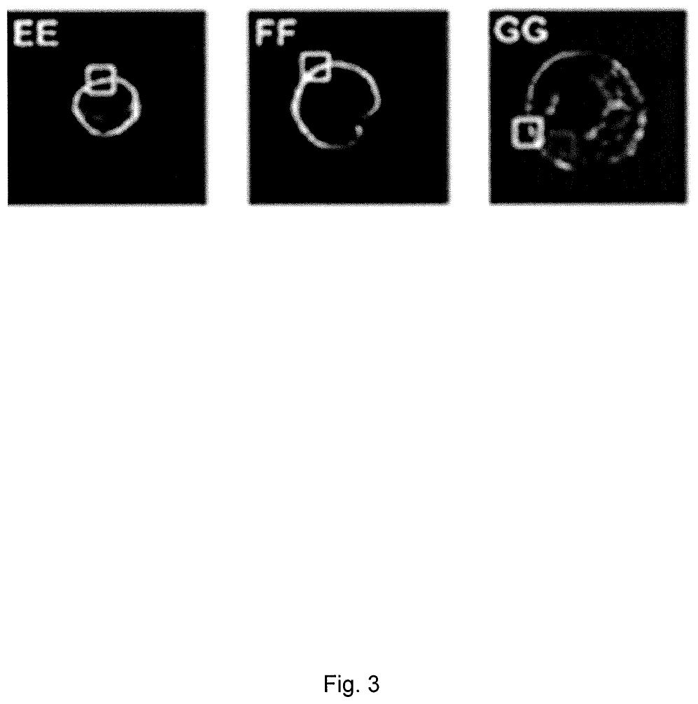

A factor that has limited the effectiveness of the iPSCDM cells is inconsistency of results for drugs with known action. The data demonstrates the fundamental basis for this problem: I_{Kr}, encoded by HERG, is inconsistently expressed at physiologically significant levels in iPSCDMs. Many cells even lack the I_{Kr}/HERG current entirely. This is not an artefact of the “immature” nature of the cells, but rather reflects the fact that HERG is not expressed as a mature cell surface protein in a very large fraction of cells in the adult heart. HERG has little or no presence on the endocardium, but is more highly expressed on the epicardium (see FIG. 3) and also has a strong apex to base gradient. iPSCDMs represent a mixture of all ventricular myocytes, and so it is not surprising that they also reflect this variability in HERG expression. This variability in expression does not reflect an “immature” state of the current iPSCDM system and is not a problem that will be solved by improved “maturation” methods. Thus, the enhanced I_{Kr} assay provided in this disclosure is expected to have a significant impact on drug safety screening.

HERG and I_{Kr} are Not Identical

The native I_{Kr} current has many complexities of its subunit composition, which are not fully reconstituted in HERG expressed in heterologous systems. Most importantly, there are actually two HERG isoforms: HERG1a and HERG1b. The rules which govern heteromultimerization are not known. However, it is known that the two isoforms can differ significantly in drug affinity. Native I_{Kr} currents are also reported to differ in their drug binding properties from cloned channels in heterologous systems. Prior research on HERG as the molecular basis for I_{Kr} has noted this discrepancy and concluded HERG current is not blocked by drugs that specifically block I_{Kr} in cardiac myocytes. The research further concluded that the data indicated that HERG proteins form I_{Kr} channels, but that an additional subunit may be required for drug sensitivity. Later, this very large and functionally significant difference was shown to cause preferential open channel block in the cloned channels which is distinct from I_{Kr}. Thus, the prediction of arrhythmogenic potential will be more accurate for I_{Kr} in a cardiac cell type in which the molecular basis is more faithfully recapitulated. Furthermore, specific protocols for the native current are designed to address issues such as use-dependence which can change apparent affinity by orders of magnitude.

The following description provides a non-limiting description of various steps of this disclosure.

Measuring I_{KrA}, I_{KrB} and I_{KrA/B}

Step 1: Provide Uniform Measureable I_{Kr} Expression of Both Isoforms

This portion of the process deals with the major problem of inconsistent expression, and even absence, of the repolarizing current I_{Kr} in iPSCDCMs. I_{Kr} is the major target for pro-arrhythmic effects and therefore cannot simply be replaced electronically. An approach of this disclosure involves modifying HERG expression by targeting a splice site in the C-terminal of HERG. This site introduces a premature truncation, a non-functional channel, and trafficking of HERG protein into cell recycling pathways.

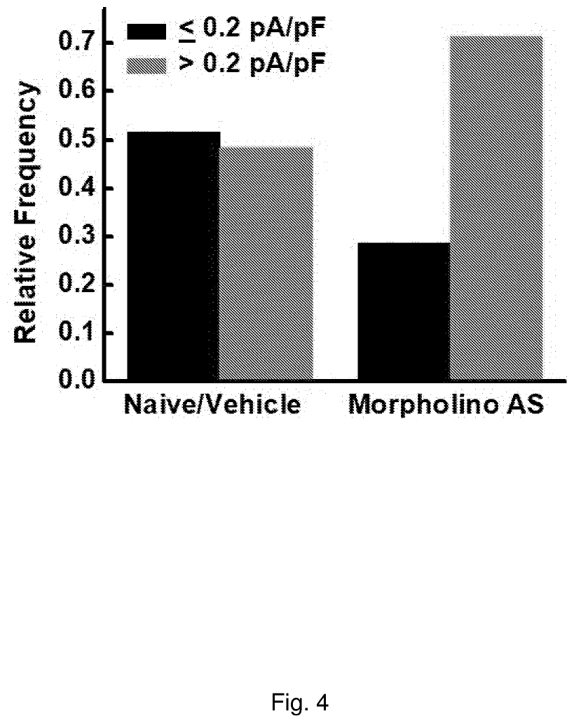

Our approach is based on studies showing that morpholino anti-sense (AS) oligonucleotides increased HERG expression in HEK293 cells. We examined the ability of these endoporter (Endo-Porter delivery system) transfected morpholino AS oligonucleotides to increase I_{Kr} expression in human iPSCDCMs. The morpholino AS oligonucleotide Endo-Porter, is a proprietary weak-base amphiphilic peptide that was designed to deliver non-ionic substances into the cytosol/nuclear compartment of cells by an endocytosis-mediated process. Following a 48 hour treatment with morpholino AS oligonucleotide (alpha2NS 05Z) significant increase (p<0.05) in I_{Kr} expression was observed compared to naïve/vehicle and an inverse morpholino AS oligonucleotide control (MPL ZN9DSZ INV) (Morpholino AS oligonucleotide (n=28): 0.29±0.04 pA/pF, INV control (n=7): 0.11±0.08 pA/pF). The increase in current density was due to a significant shift in the frequency of cells expressing higher I_{Kr} current densities in the morpholino AS oligonucleotide-treated cells. In naïve/vehicle treated cells we observed a near equal frequency of cells expressing current densities <0.2 pA/pF and >0.2 pA/pF. However, following morpholino AS oligonucleotide treatment cells expressing current densities >0.2 pA/pF increased to 71% p<0.05 as shown in FIG. 4.

The exemplary morpholino AS was identified through an understanding of the expression of the mRNA derived from the KCNH2 gene. The alternative processing of KCNH2 pre-mRNA is regulated by the relative efficiencies of RNA splicing and polyadenylation events. There are two alternative splice variants of the potassium channel encoded by KCNH2: KV11.1a, which is functional, and KV11.1a-USO, which is non-functional. Expression of the non-functional KV11.1a-USO is due to the presence of a specific poly(A) signal in Intron 9. The exemplary morpholino sequence is anti-sense to this specific poly(A) signal in Intron 9. The morpholino AS anneals to and eliminates this poly(A) signal in Intron 9 preventing the expression of the non-functional KV11.1a-USO and allowing for the preferential expression of the functional KV11.1a potassium channel variant. The net result is an increase in current levels for the KV11.1 potassium channel The morpholino AS and Endo-Porter, a peptide which allows for the cellular uptake of the morpholino AS, are commercially available and synthesized according to methods well known in the art.

This, for the first time, demonstrates that morpholino AS oligonucleotide treatment increases I_{Kr} expression in hiPSCDCMs and results in a higher frequency of cells expressing I_{Kr}, and expressing larger I_{Kr} current density. Increased I_{Kr} expression enables more accurate screening of drug effects on action potential parameters in iPSCDCMs resulting from inhibition of I_{Kr}. This is also of great importance in screening the effects of drugs using multi-planar patch electrophysiology instrumentation.

Step 2. Use Appropriate Patch Clamp Protocols to Isolate the I_{Kr} in Physiological Solutions

The above procedure increases the number of usable cells, but quality control includes monitoring these current densities. The following protocols establish a quality control procedure of this disclosure.

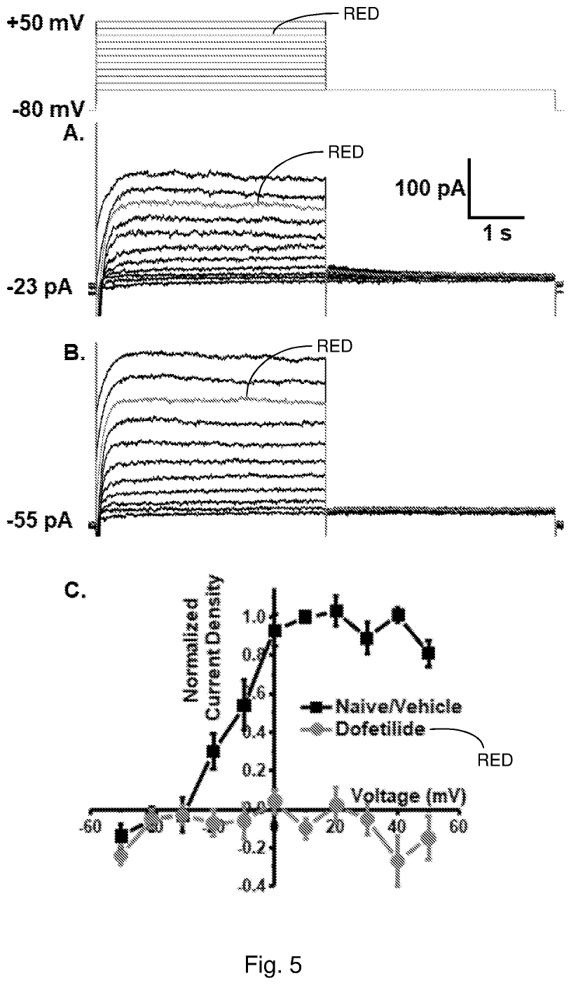

This representative protocol used commercially-available human iPSC-derived cardiac myocytes (iPSC-CMs). Electrophysiological measurements were performed in the whole-cell voltage-clamp ruptured patch configuration. This protocol also used Amphoterecin B perforated patch and saw no systematic differences between I_{Kr} currents using either method. In ruptured patch experiments, electrode solution contained (in mM): 10 NaCl, 140 KCl, MgSO₄, 5 EGTA, 5 Mg-ATP, 5 Tris creatine phosphate, 0.3 Tris-GTP, 10 HEPES, pH 7.2 (with KOH). In both, the configurations bath solution contained (in mM): 137, 4 KCl, 1.8 CaCl₂, 1 MgCl₂, 10 glucose, 10 HEPES, pH 7.4 (with NaOH). Cells were clamped at −80 mV with currents recorded over a 4 s voltage step from the holding potential to potentials between −80 and 50 mV in increments of 10 mV (P1) followed by a single voltage step to −50 mV (P2). FIG. 5A shows I_{Kr} activation in vehicle-treated iPSCDCMs. P2 tail current displayed increasing outward current with increasingly depolarizing P1 pulse. The outward P2 tail current was completely blocked after perfusion with saturating concentrations (0.3-3 μM) of dofetilide indicating that the P2 tail current consisted of well-separated I_{Kr}.

Step 3: Analyze I_{Kr} Behavior

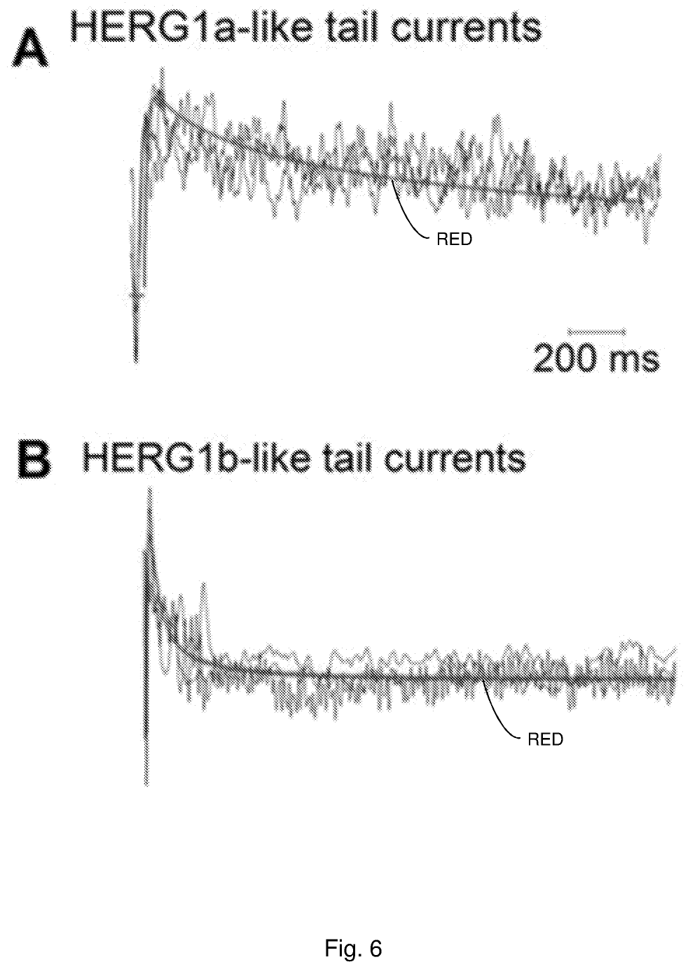

Native cardiac I_{Kr} has two distinct populations. The dofetilide-sensitive analysis of I_{Kr} tail currents in iPSCDCMs documented that the currents came primarily as one of two types. Most acutely isolated adult human cardiac myocyte data on I_{Kr} also has two similar exponential components to their deactivation.)A very slowly deactivating tail current that is essentially identical to that observed for HERG1a currents, or a very rapidly deactivating I_{Kr} that was essentially identical to HERG1b, as shown in FIGS. 6A and 6B. Analysis of a population of iPSCDCM cells showed two distinct populations as a tightly spaced set of cells characterized by a single fast time constant and a second population with a very long time constant of deactivation as shown by the histogram in FIG. 5. These were determined by fitting freely varying single and bi-exponential processes to the data. Degree of block of each of these components is determined by the changes in amplitude of each component following drug application as shown in FIG. 5C.

Step 4: Examine Dynamic Changes in Action Potential Parameters

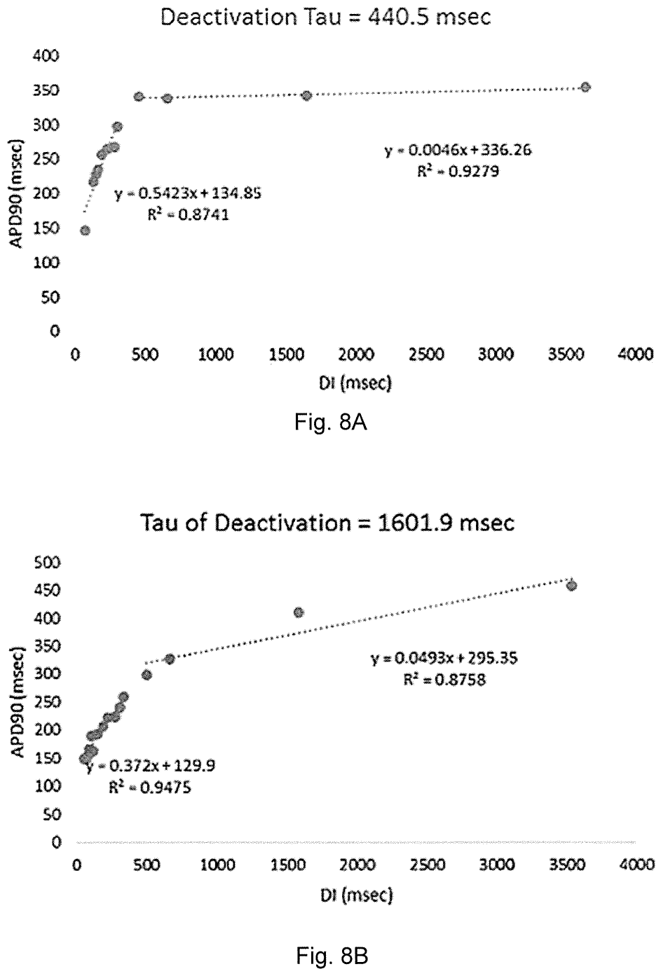

The existence of two kinetically distinct signatures suggest that the different types of cells will have different contributions of I_{Kr} to the action potential. These differences were probed for by examining the relationship between restitution and I_{Kr} and it was determined that distinct I_{Kr} deactivation is correlated with distinct restitution curves, as shown in FIG. 8. Given their distinct kinetics and correspondence with HERG1a and HERG1b, it is likely that they also have differing pharmacological profiles. Thus, characterizing these native I_{Kr} types in the context of both drug affinity and action potential behavior will lead to more accurate conclusions concerning arrhythmogenic potential than those obtained from cloned channels alone.

These data demonstrate that drug response in the action potential is strongly dependent upon the presence, size, and type of I_{Kr} present in the target iPSCDCM under study. This means that measurement protocols are desired to determine these properties during the course of an experiment so that data can be interpreted appropriately. This is done under normal physiologically relevant conditions so that action potential measurements are possible.

Assessment of Proarrhythmic Potential

It will be understood by those skilled in the art, that in embodiments, arrhythmic potential can be assessed using any of several standard techniques generally applied in the field. These include, but are not limited to: (1) Assessment of degree of I_{Kr} block relative to the therapeutic dose range; (2) Action potential prolongation; (3) Rate of occurrence of spontaneous or induced early after depolarizations; (4) Rate of occurrence of delayed after depolarizations; (5) Steepness of Restitution curves; and (6) Action potential shape changes (e.g. amplitude, upstroke velocity, triangulation, J-wave related events, etc.).

Although the present disclosure has been described with respect to one or more particular embodiments, it will be understood that other embodiments of the present disclosure may be made without departing from the spirit and scope of the present disclosure. Hence, the present disclosure is deemed limited only by the appended claims and the reasonable interpretation thereof.

1. A method for analyzing a test agent to determine whether or not the test agent affects electrical activity of a cardiomyocyte, the method comprising:
i) providing an induced pluripotent stem cell (iPSC) derived cardiomyocyte (iPSC-DM) that has been modified to contain an oligonucleotide that increases iPSC-DM I_{Kr};
ii) measuring I_{Kr} and action potential of the iPSC-DM of i); subsequently
iii) contacting the iPSC-DM of i) with the test agent; and
iv) measuring I_{Kr} and action potential of the iPSC-DM that has been contacted with the test agent;
and determining a difference between the I_{Kr} and action potential of ii) and the I_{Kr} and the action potential of iv) to indicate the test agent affects the electrical activity of the cardiomyocyte, or
determining the same I_{Kr} and action potential of ii) and iv) to indicate the test agent does not adversely affect the electrical activity of the cardiomyocyte.

2. The method of claim 1, further comprising determining a value of an inward rectifying current (I_{K1}) for the iPSC-DM of ii) and I_{K1} of the iPSC-DM of iv).

3. The method of claim 2, further comprising applying the I_{K1} of claim 2 to the iPSC-DM of ii) and iv), and determining the presence or absence of pro-arrhythmic properties in the iPSC-DM of iv) relative to pro-arrhythmic properties of the iPSC-DM of ii), wherein the presence of pro-arrhythmic properties in the iPSC-DM of iv) indicates the test agent affects electrical activity of cardiomyocytes, and the absence of pro-arrhythmic properties in the iPSC-DM of iv) indicates the test agent does not affect the electrical activity of the cardiomyocytes.

4. The method of claim 3, wherein the pro-arrhythmic properties comprise any of: early after depolarizations, action potential prolongation, delayed after depolarizations, steepness of restitution, and action potential triangulations or other in-vitro indices of development of a pro-arrhythmic substrate.

5. The method of claim 1, further comprising determining I_{Kr} and action potential for a plurality of iPSC-DMs of ii) and iv) in a series of distinct samples of the iPSC-DMs.

6. The method of claim 5, further comprising determining I_{K1} for the plurality of the iPSC-DMs of ii) and iv) in a series of distinct samples of the iPSC-DMs.

7. The method of claim 6, further comprising applying the I_{K1} to the plurality of iPSC-DMs in the series of distinct samples of the iPSC-DMs.

8. The method of claim 7, further comprising determining the presence or absence of pro-arrhythmic properties in the plurality of the iPSC-DMs.

9. The method of claim 1, further comprising measuring the I_{Kr} and action potential of the iPSC-DM of ii) to characterize I_{Kr} and the action potential as either slowly deactivating-type (HERG1a-like) or rapidly deactivating-type (HERG1b-like).

10. The method of claim 9, comprising measuring the I_{Kr} and action potential of a series of distinct samples of iPSC-DMs to characterize the I_{Kr} and the action potential of iPSC-DMs as either slowly deactivating-type (HERG1a-like) or a rapidly deactivating-type (HERG1b-like).


SEQUENCE LISTING (1 sequence)

SEQ ID NO: 1 - DNA, 25 bp, Artificial Sequence
  Morpholino AS oligonucleotide
cagaacacagtagtgaatcaaaacc